(19) United States
(12) Patent Application Publication
(10) Pub. No.: US 2012/0214875 A1
(43) Pub. Date: Aug. 23, 2012

(54) PHENOXYBENZAMINE ASSAY

(76) Inventors: Nicola Christine HUGHES, Toronto (CA); Mei Li, Toronto (CA)

(21) Appl. No.: 13/402,822

(22) Filed: Feb. 22, 2012

Related U.S. Application Data
(60) Provisional application No. 61/445,496, filed on Feb. 22, 2011.

(51) Int. Cl. A61K 31/138; H01J 49/26; G01N 33/493; G01N 30/00; G01N 33/487; G01N 33/49

(52) U.S. Cl. 514/651; 73/61.43; 73/61.52; 250/282

(57) ABSTRACT

The invention provides methods for measuring concentrations of basic or neutral compounds, such as phenoxybenzamine, in biological samples, and methods for treating patients based on the compound concentrations in the biological samples from the patients.

### CROSS-REFERENCE TO RELATED APPLICATIONS

This application claims priority benefit of U.S. Provisional Patent Application No. 61/445,496, filed Feb. 22, 2011. The entire contents of that application are hereby incorporated by reference herein.

### TECHNICAL FIELD OF INVENTION

The invention is related to measurement of the concentration of a basic or neutral drug or compound, such as phenoxybenzamine, in biological samples.

### BACKGROUND OF THE INVENTION

Basic and neutral drugs such as phenoxybenzamine are of great importance to pharmacologists and toxicologists. Phenoxybenzamine (also known as (RS)—N-benzyl-N-(2-chloroethyl)-1-phenoxy-propan-2-amine) has a molecular formula of C₁₈H₂₂ClNO, a molecular weight of 303.83 and has the following structure:

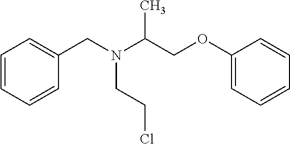

Phenoxybenzamine is used in the treatment of hypertension and its levels in blood have been the subject of inquiry. Anderson, W. H. and Stafford, D. T., Applications of capillary gas chromatography in routine toxicological analyses, Journal of High Resolution Chromatography & Chromatography Communications, 6, 247-254 (1983). Anderson and Stafford detected the presence of phenoxybenzamine in biological samples but they did not determine the concentration of phenoxybenzamine in the samples tested. In another study, an extraction method, coupled with gas chromatography and nitrogen-specific detection, provided detection limits below 200 nanograms per milliliter of blood for drugs including phenoxybenzamine. Sharp, M. E., Evaluation of a screening procedure for basic and neutral drugs: N-butyl chloride extraction and megabore capillary gas chromatography. Can. Soc. Forens. Sci. J., 19 (2): 83-101 (1986).

There are no existing methods to measure phenoxybenzamine concentrations in blood which are significantly below 200 nanograms per milliliter. The present invention provides a reliable method for quantifying very low amounts of basic or neutral compounds, such as phenoxybenzamine, in biological samples. The present invention also provides extraction solutions in which phenoxybenzamine is stable, which leads to significant relaxation of time constraints on performing an assay on phenoxybenzamine prior to degradation of phenoxybenzamine in the sample.

### SUMMARY OF THE INVENTION

The present invention is related to methods of determining concentration of basic or neutral compounds, such as phenoxybenzamine. The methods described here provide a sensitive and reliable way to measure the concentration of a basic or neutral compound such as phenoxybenzamine in biological samples, such as blood.

Provided here are methods for preparing an extract from a biological sample suitable for determining concentration of a basic or neutral compound, comprising the step of extracting the compound from the biological sample using an acidic alcohol-water mixture. In some embodiments, the basic or neutral compound is phenoxybenzamine.

Also provided are methods for determining the concentration of a basic or neutral compound (e.g., phenoxybenzamine) in a biological sample comprising the steps of (a) extracting the compound from the biological sample using an acidic alcohol-water mixture to obtain an alcohol extract comprising the compound and (b) analyzing the alcohol extract to determine the concentration of the compound. In some embodiments, the basic or neutral compound is phenoxybenzamine.

Also provided are methods for stabilizing a basic or neutral compound comprising combining a composition comprising the basic or neutral compound with an acidic alcohol-water mixture, wherein stability of the basic or neutral compound is enhanced as compared to a composition without the acidic alcohol-water mixture. In some embodiments, the composition comprises the compound (e.g., phenoxybenzamine) at a concentration of any of about 1 ng/mL to about 1 g/mL, about 20 ng/mL to about 100 mg/mL, about 20 ng/mL to about 10 mg/mL, about 50 ng/mL to about 50 mg/mL, about 20 ng/mL to about 1 mg/mL, about 20 ng/mL to about 500 μg/mL, about 20 ng/mL to about 100 μg/mL, about 20 ng/mL to about 10 μg/mL, about 20 ng/mL to about 1 μg/mL, about 20 ng/mL to about 500 ng/mL, about 20 ng/mL to about 100 ng/mL, about 50 ng/mL to about 1 mg/mL, about 50 ng/mL to about 500 μg/mL, about 50 ng/mL to about 100 μg/mL, about 50 ng/mL to about 10 μg/mL, about 50 ng/mL to about 1 μg/mL, about 50 ng/mL to about 500 ng/mL, or about 50 ng/mL to about 100 ng/mL. In some embodiments, the basic or neutral compound is phenoxybenzamine.

This invention provides a method for determining the concentration of a basic or neutral compound (e.g., phenoxybenzamine) in a biological sample comprising the steps of (a) extracting the compound from the biological sample to obtain an extract comprising the compound, wherein the extract comprises at least about 30% (e.g., at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98%) of the compound from the biological sample, and (b) analyzing the extract to determine the concentration of the compound. In some embodiments, the basic or neutral compound is phenoxybenzamine.

Another aspect of the present invention features a method for determining the concentration of a basic or neutral compound (e.g., phenoxybenzamine) in a biological sample comprising the steps of (a) extracting the compound from the biological sample to obtain an extract comprising the compound and (b) analyzing the extract to determine the concentration of the compound, wherein the method is used to detect less than about 200 nanograms (“ng”) (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng) of the compound from 1 milliliter (“mL”) of the biological sample. In some embodiments, the basic or neutral compound is phenoxybenzamine.

Another aspect of the present invention features a method for determining the concentration of a basic or neutral compound (e.g., phenoxybenzamine) in a biological sample comprising the steps of (a) extracting the compound from the biological sample to obtain an extract comprising the compound and (b) analyzing the extract to determine the concentration of the compound, wherein the method is used to detect between about 0.1 ng to about 200 nanograms (“ng”) (e.g., about 0.1 ng to about 150 ng, about 0.1 ng to about 100 ng, about 0.1 ng to about 75 ng, about 0.1 ng to about 50 ng, about 0.1 ng to about 25 ng, about 0.1 ng to about 10 ng, about 0.1 ng to about 5 ng, about 0.1 ng to about 2 ng, about 0.1 ng to about 1 ng, about 0.1 ng to about 0.75 ng, about 0.1 ng to about 0.5 ng, about 0.1 ng to about 0.25 ng, about 1 ng to about 150 ng, about 1 ng to about 100 ng, about 1 ng to about 75 ng, about 1 ng to about 50 ng, about 1 ng to about 25 ng, about 1 ng to about 10 ng, about 1 ng to about 5 ng, or about 1 ng to about 2 ng) of the compound from 1 milliliter (“mL”) of the biological sample. In some embodiments, the basic or neutral compound is phenoxybenzamine.

Any of the methods provided herein may be used to determine the concentration of a modified phenoxybenzamine, a derivative of phenoxybenzamine, a salt of phenoxybenzamine, a metabolite of phenoxybenzamine, an analog of phenoxybenzamine, a degradation product of phenoxybenzamine, or a compound which has a similar structure as phenoxybenzamine. Any of the methods provided herein may be used to determine the concentration of a secondary amine, a tertiary amine, or a β-chloroethyl form of phenoxybenzamine. Any of the methods provided herein may be used to determine the concentration of N-(1-phenoxyisopropyl)-amino-1-ethanol, N-(1-phenoxyisopropyl)-β-chloroethylamine hydrochloride, N-phenoxyisopropyl-N-benzyl-aminoethanol, N-phenoxyisopropyl-N-benzyl-2-propoxyethylamine, or di-(N-phenoxyisopropyl-N-benzyl-2-benzyloxyethyamine.

Any of the methods provided herein may be used to determine the concentration of a basic or neutral compound (e.g., phenoxybenzamine) in a biological sample. In some embodiments, the biological sample is a biological liquid sample or a biological tissue sample. In some embodiments, the biological sample is blood or urine. In some embodiments, the biological sample is an extract from biological liquid sample or biological tissue sample, such as blood serum or blood plasma. In some embodiments, the biological sample is obtained from a patient treated with phenoxybenzamine.

Another aspect of the present invention features a method for determining the concentration of a neutral or basic compound (e.g., phenoxybenzamine) in a blood plasma sample comprising the steps of (a) extracting the compound from the blood plasma sample using an acidic alcohol-water mixture to obtain an alcohol extract comprising the compound, and (c) analyzing the alcohol extract to determine the concentration of the compound. The compound may be phenoxybenzamine, a modified phenoxybenzamine, a derivative of phenoxybenzamine, a metabolite of phenoxybenzamine, an analog of phenoxybenzamine, or a compound that has similar structure as phenoxybenzamine.

The solution or solvent used to extract neutral or basic compound (such as phenoxybenzamine) from biological sample may be an alcohol-water mixture. In some embodiments, the alcohol-water mixture is an acidic alcohol-water mixture comprising alcohol and acid. The acid may be any organic acid or inorganic acid, e.g., trifluoroacetic acid (“TFA”), hydrochloric acid, phosphoric acid, or nitric acid. The acidic alcohol-water mixture may comprise less than about 1% TFA (e.g., less than about 0.75% TFA, less than about 0.5% TFA, less than about 0.2% TFA, less than about 0.1% TFA, less than about 0.075% TFA, or less than about 0.05% TFA). The acidic alcohol-water mixture may comprise about 0.1% TFA.

The alcohol used in the alcohol-water mixture may be any alcohol. The alcohol may be methanol, ethanol, n-propanol, isopropanol, 1-butanol, 2-butanol, sec-butanol or tert-butanol. The alcohol-water mixture may comprise at least about 10% (e.g., at least about 20%, at least about 40%, at least about 60%, at least about 80%, at least about 90%, or at least about 95%) alcohol. The alcohol-water mixture may comprise about 80% methanol or about 80% ethanol.

The step of analyzing an extract comprising a neutral or basic compound (such as phenoxybenzamine) from a biological sample to determine the concentration of the compound may be performed using liquid chromatography. In some embodiments, the analysis step is performed using high performance liquid chromatography using reverse phase chromatographic conditions. In some embodiments, the analysis step is performed using liquid chromatography coupled with mass spectrometry (“LC-MS/MS”), e.g., liquid chromatography coupled with tandem mass spectrometry. In some embodiments, the analysis step is performed using high performance liquid chromatography coupled with UV/VIS detection. In some embodiments, the analysis step is performed using ultra performance liquid chromatography. In some embodiments, the analysis step is performed using ultra performance liquid chromatography coupled with tandem mass spectrometry (UPLC-MS/MS).

Also within the scope of this invention is a method of treating a patient, comprising the steps of (a) administering phenoxybenzamine to the patient, (b) measuring the concentration of phenoxybenzamine in a biological sample obtained from the patient, and (c) based on the concentration of phenoxybenzamine in the biological sample, adjusting the amount of a drug (which may be phenoxybenzamine or another drug) already being administered to the patient, or administering a new drug to the patient, in order to achieve a therapeutic benefit in the patient.

Also within the scope of this invention is a method of treating a patient comprising the steps of (a) administering phenoxybenzamine to the patient, (b) measuring the concentration of phenoxybenzamine in a biological sample obtained from the patient, and (c) based on the concentration of phenoxybenzamine in the biological sample, adjusting the dosage of phenoxybenzamine administered to the patient in order to achieve a therapeutic benefit.

In one embodiment of the invention, the accuracy and/or selectivity of the method for determination of the concentration of a basic or neutral drug, such as phenoxybenzamine, is not significantly affected by the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol. In another embodiment of the invention, the accuracy and/or selectivity of the method for determination of the concentration of phenoxybenzamine is not significantly affected by the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol.

The details of one or more embodiments of the invention are set forth in the description below. Other features or advantages of the present invention will be apparent from the following detailed description of several embodiments and from the appended claims.

### DETAILED DESCRIPTION OF THE INVENTION

Provided here are methods for preparing an extract from a biological sample suitable for determining concentration of a basic or neutral compound, comprising the step of extracting the compound from the biological sample using an acidic alcohol-water mixture. In some embodiments, the basic or neutral compound is phenoxybenzamine.

Also provided here are methods for determining the concentration of a basic or neutral compound (e.g., phenoxybenzamine) in a biological sample comprising the steps of (a) extracting the compound from the biological sample using an acidic alcohol-water mixture to obtain an alcohol extract comprising the compound and (b) analyzing the alcohol extract to determine the concentration of the compound. In some embodiments, the basic or neutral compound is phenoxybenzamine.

The methods provided may be used to detect very low amounts of basic or neutral compounds in biological samples, for example, to detect less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng of the compound from 1 milliliter (“mL”) of the biological sample. In some embodiments, the basic or neutral compound is phenoxybenzamine.

Any of the methods provided herein may be used to determine the concentration of basic or neutral compounds (e.g., phenoxybenzamine) in biological samples. Biological samples may be biological liquid samples or biological tissue samples. Biological samples may be blood samples or urine samples. Biological samples may be extracts from biological liquid samples or biological tissue samples. Biological samples may be treated or processed prior to performance of the extraction step (for example, prior to performance of the extraction of compound using an alcohol-water mixture). To be suitable for the extraction step, biological samples may be processed to remove substances that might interfere with the extraction step, reduce the extraction efficiency, or affect the later analyzing step for determining the concentration by, e.g., liquid chromatography. In some embodiments, the basic or neutral compound is phenoxybenzamine.

Any of the methods provided herein may be used to determine the concentration of phenoxybenzamine, its derivatives, its metabolites, its analogs, its degradation products, or compounds that have similar structures as phenoxybenzamine. Also provided herein are methods for determining the concentration of a modified phenoxybenzamine, such as chemically modified phenoxybenzamine. Also provided herein are methods for determining the concentration of any salt form of phenoxybenzamine. Any of the methods provided herein may be used to determine the concentration of a secondary amine, a tertiary amine, or a β-chloroethyl form of phenoxybenzamine. Any of the methods provided herein may be used to determine the concentration of N-(1-phenoxyisopropyl)-amino-1-ethanol, N-(1-phenoxyisopropyl)-β-chloroethylamine hydrochloride, N-phenoxyisopropyl-N-benzyl-aminoethanol, N-phenoxyisopropyl-N-benzyl-2-propoxyethylamine, or di-(N-phenoxyisopropyl-N-benzyl-2-benzyloxyethyamine.

Also disclosed herein are methods of extracting a basic or neutral compound (e.g., phenoxybenzamine) from a biological sample, wherein at least about 30% (e.g., at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 98%) of the compound in the biological sample is recovered by the extraction step. In some embodiments, the basic or neutral compound is phenoxybenzamine.

Also disclosed herein are methods of determining the concentration of a basic or neutral compound (e.g., phenoxybenzamine) from a biological sample, wherein the methods are used to detect less than about 200 nanograms (“ng”) (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng) of the compound from 1 milliliter (“mL”) of the biological sample. In some embodiments, the basic or neutral compound is phenoxybenzamine.

In any of the methods provided herein, a solution is used to extract the basic or neutral compound (e.g., phenoxybenzamine) from the biological sample. In some embodiments, the solution is an alcohol-water mixture. In some embodiments, the solution is an acidic alcohol-water mixture. In some embodiments, the alcohol-water mixture comprises at least one alcohol and at least one acid. The acid may be an organic or inorganic acid, e.g., trifluoroacetic acid (“TFA”), nitric acid, hydrochloric acid, formic acid, or phosphoric acid. In some embodiments, the acid is TFA. In some embodiments, the acidic alcohol-water mixture comprises any of about 0.01% to about 5% acid (e.g., TFA) (vol/vol), about 0.05% to about 2% acid (e.g., TFA), about 0.05% to about 1% acid (e.g., TFA), about 0.05% to about 0.5% acid (e.g., TFA), or about 0.05% to about 0.2% acid (e.g., TFA). In some embodiments, the acidic alcohol-water mixture comprises at least about any of 0.01% acid (e.g., TFA), 0.02% acid (e.g., TFA), 0.05% acid (e.g., TFA), 0.1% acid (e.g., TFA), 0.2% acid (e.g., TFA), 0.5% acid (e.g., TFA), or 1% acid (e.g., TFA). In some embodiments, the acidic alcohol-water mixture comprises less than about any of 1% acid (e.g., TFA), 0.75% acid (e.g., TFA), 0.5% acid (e.g., TFA), 0.2% acid (e.g., TFA), 0.1% acid (e.g., TFA), 0.075% acid (e.g., TFA), or 0.05% acid (e.g., TFA). In some embodiments, the acidic alcohol-water mixture comprises about any of 0.01% acid (e.g., TFA), 0.02% acid (e.g., TFA), 0.05% acid (e.g., TFA), 0.1% acid (e.g., TFA), 0.2% acid (e.g., TFA), 0.5% acid (e.g., TFA), or 1% acid (e.g., TFA). In some embodiments, the acid is TFA. In some embodiments, the basic or neutral compound is phenoxybenzamine.

In some embodiments, the alcohol-water mixture comprises at least an alcohol, wherein the alcohol is methanol, ethanol, n-propanol, isopropanol, 1-butanol, 2-butanol, sec-butanol or tert-butanol. In some embodiments, the alcohol-water mixture may comprise at least about 10% (vol/vol) (e.g., at least about 20%, at least about 40%, at least about 60%, at least about 80%, at least about 90%, or at least about 95%) alcohol. In some embodiments, the alcohol-water mixture comprises any of about 10% to about 95%, about 20% to about 90%, about 40 to about 80%, or about 50% to about 80% alcohol. In some embodiments, the alcohol-water mixture comprises about any of 10%, 20%, 30%, 40%, 50%, 60%, 70%, 80%, 90% alcohol. In some embodiments, the alcohol is methanol or ethanol. In some embodiments, the alcohol-water mixture comprises about 80% methanol or about 80% ethanol.

Provided herein are methods of determining the concentration of a basic or neutral compound (e.g., phenoxybenzamine) in a biological sample. In some embodiments, the biological sample comprises less than about 200 ng (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng) of the compound (e.g., phenoxybenzamine) per 1 mL of the biological sample. In some embodiments, the basic or neutral compound is phenoxybenzamine.

Provided herein are methods of determining the concentration of a basic or neutral compound (e.g., phenoxybenzamine) in a biological sample. In some embodiments, the biological sample comprises between about 0.1 ng to about 200 nanograms (“ng”) (e.g., about 0.1 ng to about 150 ng, about 0.1 ng to about 100 ng, about 0.1 ng to about 75 ng, about 0.1 ng to about 50 ng, about 0.1 ng to about 25 ng, about 0.1 ng to about 10 ng, about 0.1 ng to about 5 ng, about 0.1 ng to about 2 ng, about 0.1 ng to about 1 ng, about 0.1 ng to about 0.75 ng, about 0.1 ng to about 0.5 ng, about 0.1 ng to about 0.25 ng, about 1 ng to about 150 ng, about 1 ng to about 100 ng, about 1 ng to about 75 ng, about 1 ng to about 50 ng, about 1 ng to about 25 ng, about 1 ng to about 10 ng, about 1 ng to about 5 ng, or about 1 ng to about 2 ng) of the compound per 1 milliliter (“mL”) of the biological sample.

In one embodiment, the biological sample used to measure the concentration of a basic or neutral drug or compound (e.g., phenoxybenzamine) is a blood sample. Plasma is obtained from whole blood using any appropriate method known to those skilled in the art, for example, by centrifugation or by allowing the sample to sit for a period of time. Anticoagulant agents may be used. In some embodiments, the basic or neutral compound is phenoxybenzamine.

Also provided herein are methods of extracting a basic or neutral drug or compound from a biological sample, wherein the extraction is performed by solid phase extraction, solid phase micro extraction, or liquid/liquid extraction. In some embodiments, the basic or neutral compound is phenoxybenzamine.

Also disclosed herein are methods of extracting a basic or neutral drug or compound from a biological sample by solid phase extraction using an acidic alcohol-water mixture. In some embodiments, the solid phase extraction is performed using Oasis® HLB cartridge (available from Waters), STRATA-X functionalized Polymeric SPE (available from Phenomenex), or SampliQ WAX (available from Agilent). In some embodiments, the basic or neutral compound is phenoxybenzamine.

For purposes of measuring the concentration of a basic or neutral compound (e.g., phenoxybenzamine) from a blood sample, the blood sample may be stored for no longer than about 20 hours (e.g., no longer than about 10 hours, no longer than about 5 hours, or no longer than about 2 hours) prior to separating the plasma from the blood sample. The blood sample may be stored at room temperature or at a temperature between about 0° C. and about 25° C. (e.g., between about 0° C. and about 15° C., between about 0° C. and about 10° C., or between about 0° C. and about 8° C., or between about 0° C. and about 5° C., or between about 1° C. and about 15° C., between about 1° C. and about 10° C., or between about 1° C. and about 8° C., or between about 1° C. and about 5° C.) prior to separating the plasma from the blood sample. In some embodiments, the basic or neutral compound is phenoxybenzamine.

After the compound is extracted using an acidic alcohol-water mixture, the extract may be stored for no more than about 500 hours (or, in other embodiments, no more than about 300 hours, no more than about 200 hours, or no more than about 110 hours) prior to being analyzed. The extract may be stored at room temperature or at a temperature between about 0° C. and about 25° C. (e.g., between about 0° C. and about 15° C., between about 0° C. and about 10° C., or about 0° C. and about 8° C., or about 0° C. and about 5° C., between about 3° C. and about 15° C., between about 3° C. and about 11° C.) prior to analyzing the extract.

An analysis method is provided to determine the concentration of a basic or neutral compound, such as phenoxybenzamine. The analysis method may be any method which is sensitive to provide detection limits of less than about 200 ng (from 1 mL of biological sample) (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng). In some embodiments, the basic or neutral compound is phenoxybenzamine.

In some embodiments, the analysis method is liquid chromatography, e.g., high performance liquid chromatography. In some embodiments, the analysis method is high performance liquid chromatography using reverse phase chromatographic conditions. In some embodiments, the analysis method is liquid chromatography coupled with mass spectrometry (“LC-MS/MS”), e.g., liquid chromatography coupled with tandem mass spectrometry. In some embodiments, the analysis method is high performance liquid chromatography coupled with UV/VIS detection. In some embodiments, the analysis method is ultra performance liquid chromatography. In some embodiments, the analysis method is ultra performance liquid chromatography coupled with tandem mass spectrometry (UPLC-MS/MS).

Regarding any of the methods described herein, the biological sample may be obtained from a patient treated with phenoxybenzamine or other basic or neutral drugs. A patient may be treated with phenoxybenzamine or other basic or neutral drugs for hypertension or other diseases. In some embodiments, phenoxybenzamine is administered to the patient to treat hypertension associated with pheochromocytoma. In some embodiments, phenoxybenzamine is administered to the patient to manage hypertensive occurrences associated with pheochromocytoma. In some embodiments, phenoxybenzamine is administered to the patient to treat urinary retention due to neurogenic bladder. In some embodiments, phenoxybenzamine is administered to the patient for short-term management of urinary retention due to neurogenic bladder. In some embodiments, phenoxybenzamine is administered to the patient to treat benign prostatic hypertrophy. In some embodiments, phenoxybenzamine is administered to the patient for short term treatment of benign prostatic hypertrophy. In some embodiments, phenoxybenzamine is administered for short term treatment of benign prostatic hypertrophy in a patient awaiting surgery. In some embodiments, phenoxybenzamine is administered to a patient with inoperable benign hypertrophy.

Also within the scope of the invention is a method of treating a patient comprising the steps of (a) administering phenoxybenzamine to the patient, (b) measuring the concentration of phenoxybenzamine in a biological sample obtained from the patient, and (c) based on the concentration of phenoxybenzamine in the biological sample, adjusting the amount of a drug (which may be phenoxybenzamine or another drug) already being administered to the patient, or administering a new drug to the patient, in order to achieve a therapeutic benefit in the patient. Measurement of the concentration of phenoxybenzamine in a biological sample from a patient may indicate that the concentration is higher or lower than the desired level, and the amount of the phenoxybenzamine given to the patient may be adjusted (i.e., the dosage may be increased or decreased) in order to provide a therapeutic benefit. Alternatively, other drugs which may be beneficial to the patient may be administered.

Also within the scope of the invention is a method of treating a patient comprising the steps of (a) administering phenoxybenzamine to the patient, (b) measuring the concentration of phenoxybenzamine in a biological sample obtained from the patient, and (c) based on the concentration of phenoxybenzamine in the biological sample, adjusting the dosage of phenoxybenzamine administered to the patient in order to achieve a therapeutic benefit. Measurement of the concentration of phenoxybenzamine in a biological sample from a patient may indicate that the concentration is higher or lower than the desired level, and the amount of the phenoxybenzamine given to the patient may be adjusted (i.e., the dosage may be increased or decreased) in order to provide a therapeutic benefit.

The therapeutic benefit may comprise any effect that would improve the health of a patient. The therapeutic benefit may comprise lowering a patient's blood pressure. Alternatively, if the blood pressure of a patient has been lowered excessively, the therapeutic benefit may comprise raising the patient's blood pressure.

Also provided are methods for stabilizing a basic or neutral compound comprising combining a composition comprising the basic or neutral compound with an acidic alcohol-water mixture, wherein stability of the basic or neutral compound is enhanced as compared to a composition without the acidic alcohol-water mixture. In some embodiments, the composition comprises the compound (e.g., phenoxybenzamine) at a concentration of any of about 1 ng/mL to about 1 g/mL, about 20 ng/mL to about 100 mg/mL, about 20 ng/mL to about 100 mg/mL, about 50 ng/mL to about 50 mg/mL, 20 ng/mL to 1 mg/mL, or about 50 ng/mL to 1 mg/mL. In some embodiments, the composition comprises the compound (e.g., phenoxybenzamine) at a concentration of at least about any of 1 ng/mL, 20 ng/mL, 50 ng/mL, 100 ng/mL, 500 ng/mL, 1 mg/mL, 5 mg/mL, 10 mg/mL, 50 mg/mL, 100 mg/mL, 500 mg/mL, or 1000 mg/mL. In some embodiments, the composition comprises the compound (e.g., phenoxybenzamine) at a concentration of about any of 1 ng/mL, 20 ng/mL, 50 ng/mL, 100 ng/mL, 500 ng/mL, 1 mg/mL, 5 mg/mL, 10 mg/mL, 50 mg/mL, 100 mg/mL, 500 mg/mL, or 1000 mg/mL “Stabilizing a basic or neutral compound” means that the stability of the basic or neutral compound is improved. The stability of a basic or neutral compound is improved when the reduction of the measured concentration of the basic or neutral compound is less than about any of 10%, 5%, 2%, or 1% over a duration in time at room temperature, 4° C., or 0° C. In some embodiments, the duration in time is about any of 1 hour, 2 hours, 5 hours, 10 hours, 20 hours, 50 hours, 80 hours, 100 hours, 110 hours, or 120 hours. In some embodiments, the basic or neutral compound is phenoxybenzamine.

Also provided herein are compositions comprising phenoxybenzamine and alcohol. In some embodiments, phenoxybenzamine is extracted from a biological sample. In some embodiments, phenoxybenzamine is extracted from a biological sample comprising less than about 200 ng (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng) of phenoxybenzamine per 1 mL of the biological sample. In some embodiments, phenoxybenzamine in the composition is less than about 200 ng (e.g., less than about 150 ng, less than about 100 ng, less than about 75 ng, less than about 50 ng, less than about 25 ng, less than about 10 ng, less than about 5 ng, less than about 2 ng, less than about 1 ng, less than about 0.75 ng, less than about 0.5 ng, less than about 0.25 ng, or less than about 0.1 ng). In some embodiments, the composition comprises at least about 10% (vol/vol) (e.g., at least about 20%, at least about 40%, at least about 60%, at least about 80%, at least about 90%, or at least about 95%) alcohol. In some embodiments, the composition further comprises less than about 1% TFA (vol/vol) (e.g., less than about 0.75% TFA, less than about 0.5% TFA, less than about 0.2% TFA, less than about 0.1% TFA, less than about 0.075% TFA, or less than about 0.05% TFA).

The accuracy of the method for determination of the concentration of a basic or neutral drug, such as phenoxybenzamine, is not significantly affected by the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol, when these compounds are present at typical concentrations in the biological sample (i.e., concentrations within medically acceptable ranges in humans or animals for the compounds; or, in other embodiments, in amounts plus or minus about 99%, about 90%, about 75%, or about 50% of the amount of the basic or neutral drug, such as phenoxybenzamine, present). By “not significantly affected” is meant that the presence of one or more of the compounds indicated causes an inaccuracy in measurement of less than about 10%, less than about 5%, less than about 2%, less than about 1%, or less than the standard deviation of measurement in the absence of one or more of the compounds indicated.

The selectivity of an assay refers to “the extent to which the method can be used to determine particular analytes in mixtures or matrices without interferences from other components of similar behavior” (Vessman, J et al., Pure Appl. Chem., 73(8):1381-1386 (2001)). The selectivity of the method for determination of the concentration of a basic or neutral drug, such as phenoxybenzamine, is not significantly affected by the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol, when these compounds are present at typical concentrations in the biological sample (i.e., concentrations within medically acceptable ranges in humans or animals for the compounds; or, in other embodiments, in amounts plus or minus about 99%, about 90%, about 75%, or about 50% of the amount of the basic or neutral drug, such as phenoxybenzamine, present). By “not significantly affected” is meant that the presence of one or more of the compounds indicated causes less than about 20%, less than about 10%, less than about 5%, less than about 2%, less than about 1%, or less than about 0.1% interference in the measurement of a basic or neutral drug, such as phenoxybenzamine at a concentration equivalent to the lower limit of quantitation (“LLOQ”) of the basic or neutral drug, such as phenoxybenzamine, in the absence of one or more of the compounds indicated.

As is understood by one of skill in the art, reference to “about” a value or parameter herein includes (and describes) embodiments that are directed to that value or parameter per se. For example, description referring to “about X” includes description of “X.”

### EXAMPLES

### Example 1

### Determining Concentration of Phenoxybenzamine in a Plasma Sample

Phenoxybenzamine and the internal standard phenoxybenzamine-d5 were used in the studies. The molecular structure and molecular weight of phenoxybenzamine and phenoxybenzamine-d5 are shown in Table I below.

TABLE I

| | |
|---|---|
| Phenoxybenzamine molecular formula C₁₈H₂₂ClNO formula weight 303.83 | 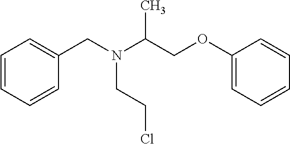 |
| Phenoxybenzamine-d5 molecular formula C₁₈H₁₇ClNOD₅ formula weight 308.86 | 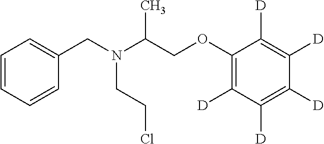 |

Phenoxybenzamine and the internal standard phenoxybenzamine-d5 were extracted from 0.2 mL of human plasma, using EDTA (potassium K₂ salt) as the anticoagulant and containing 1.3% of H₃PO₄ as stabilizer, by solid phase extraction into 0.8 mL of 0.1% TFA in 80% methanol, followed by dilution with 0.4 mL of 0.1% TFA. An aliquot of this extract was injected into a high performance liquid chromatography system and detected using an API 3000 tandem mass spectrometer with HSID interface and quantitated using peak area ratio method. Method sensitivity and selectivity were achieved by detecting distinct precursor to product ion mass transitions for phenoxybenzamine (304.1→120.0) and the internal standard phenoxybenzamine-d5 (309.1→120.0) at defined retention times under reverse chromatographic conditions.

Extracting Phenoxybenzamine from a Plasma Sample

Blood plasma was separated from whole blood. The plasma samples should be protected from light and may be stored at −70° C. for use. The plasma samples were thawed in cold tap water and analyzed along with a set of standard samples. All samples were vortexed for about 20 seconds, centrifuged at 3000 rpm for 5 minutes at 4° C.±4° C., and placed in an ice-water bath. 200 uL of plasma was added to polypropylene tubes and kept in ice-water bath. 100 uL of internal standard working solution (40 ng/mL of phenoxybenzamine-d5) was added into all the tubes except those labeled with “BLANK.” 100 uL of 0.1 N HCl in 20% methanol was added to those labeled with BLANK and the tubes were vortexed for about 20 seconds. 400 uL of 1.7% phosphoric acid was added into all tubes. The tubes were vortexed for about 40 seconds and left in an ice-water bath. The Oasis™ HLB 1 cc (30 mg) cartridges (Waters) were washed first with 700 uL methanol and then with 700 uL water. The samples prepared above were then transferred into the cartridges which were then centrifuged at 1000 rpm for 2 minutes at 4° C.±4° C. Cartridges were then first washed with 700 uL water and then washed with 700 uL of 0.1% TFA in 40% methanol. Cartridges were then eluted twice with 400 μL of 0.1% TFA in 80% methanol, and all of the eluates were collected and diluted with 400 μL of 0.1% TFA. The extracted samples were then ready for analysis.

### Detection Limit of Phenoxybenzamine

The limit of detection for phenoxybenzamine was determined by injecting a series of dilutions of a neat standard solution. The result is summarized in Table II.

TABLE II
Limit of detection for phenoxybenzamine.

| | |
|---|---|
| Concentration of | 0.002750 |
| Injected Solution | |
| (ng/mL) | |
| Injected volume (uL) | 5.0 |
| Amount on column | 0.01 |
| (pg) | |
| Signal to noise ratio | 3.9 |
| (n = 3) | 4.5 |
| | 3.3 |
| Mean | 3.9 |

Limit of detection was determined by injecting diluted reference LLOQ solution in triplicate until a response equivalent to a signal to noise ratio of approximately 3:1 was achieved. The original reference LLOQ solution includes 0.033 ng/mL phenoxybenzamine.

### Determination of Recovery Percentages

The standard and quality control (“QC”) samples were prepared in human plasma, containing EDTA (potassium K₂ salt) as an anti-coagulant and containing 1.3% phosphoric acid (H₃PO₄) as stabilizer, which had been tested and found to be free of interference at the retention times of the analytes. The standards, QC samples, and blank human plasma were transferred into the appropriately labeled polypropylene tubes for extraction. The tubes were protected from light during storage.

The volume of spiking solutions and blank plasma (in the same proportion) may be adjusted as needed. Before being used for spiking, blank plasma was acidified by adding 15 uL of 85% H₃PO₄ to each mL of blank plasma and mixed well; the same treatment was applied to all experiments involving plasma. The samples were stored on an ice-water bath during the preparation of the plasma samples.

TABLE IIIa

| | Peak Area | | | |
|---|---|---|---|---|
| | | Neat in | | |
| | | Extracted Blank | | |
| Sample | Extracted | Plasma | Average of Neat | Recovery % |
| QC LOW | 21367 | 22450 | 23036 | 92.8 |
| | 20255 | 23561 | | 87.9 |
| | 21287 | 23097 | | 92.4 |
| | 20103 | | | 87.3 |
| | 20506 | | | 89.0 |
| | 20297 | | | 88.1 |
| QC MED | 163224 | 177482 | 179515 | 90.9 |
| | 167135 | 180719 | | 93.1 |
| | 161472 | 180344 | | 89.9 |
| | 161307 | | | 89.9 |
| | 161835 | | | 90.2 |
| | 164875 | | | 91.8 |
| QC HIGH | 635476 | 681822 | 685895 | 92.6 |
| | 641844 | 694212 | | 93.6 |
| | 650051 | 681650 | | 94.8 |
| | 642638 | | | 93.7 |
| | 663243 | | | 96.7 |
| | 632954 | | | 92.3 |

| | |
|---|---|
| Overall % Recovery | 91.5 |
| SD | 2.5 |
| % CV | 2.8 |

TABLE IIIb

| | Peak Area | | | |
|---|---|---|---|---|
| | | Neat in | | |
| | | Extracted Blank | | |
| Sample | Extracted | Plasma | Average of Neat | Recovery % |
| QC LOW | 509049 | 537011 | 546150 | 93.2 |
| | 504045 | 556521 | | 92.3 |
| | 529749 | 544917 | | 97.0 |
| | 497374 | | | 91.1 |
| | 532070 | | | 97.4 |
| | 506185 | | | 92.7 |
| QC MED | 505760 | 515163 | 533242 | 94.8 |
| | 521003 | 545846 | | 97.7 |
| | 508762 | 538718 | | 95.4 |
| | 522629 | | | 98.0 |
| | 513909 | | | 96.4 |
| | 511568 | | | 95.9 |
| QC HIGH | 506589 | 549344 | 540576 | 93.7 |
| | 512853 | 546240 | | 94.9 |
| | 515765 | 526145 | | 95.4 |
| | 508277 | | | 94.0 |
| | 535724 | | | 99.1 |
| | 498665 | | | 92.2 |

| | |
|---|---|
| Overall % Recovery | 95.1 |
| SD | 2.3 |
| % CV | 2.4 |

Table III. Recovery percentages of phenoxybenzamine (Table IIIa) and internal standard phenoxybenzamine-d5 (Table IIIb). The % Coefficient of Variation of recovery is calculated for each analyte and internal standard(s) individually.

The “Recovery QCs” were prepared by extracting QCs at three concentrations (QC LOW: 0.601 ng/mL; QC MED: 4.808 ng/mL; QC HIGH: 19.232 ng/mL) in replicates of six as per method. The “Reference QCs” were prepared by extracting aliquots of blank plasma containing 1.3% H₃PO₄ (three replicates per level) as per method and evaporated to dryness at 45° C.±4° C. after elution and reconstituted in 1200 uL of appropriate QC neat solution at each level (QC LOW, QC MED & QC HIGH).

QC Neat Solutions were prepared by adding phenoxybenzamine and phenoxybenzamine-d5 into a solution containing 9.95 mL of 0.1% TFA in 80% methanol, then 5 mL of 0.1% TFA was added and mixed well.

The QC High Neat Solution contained 3.205 ng/mL phenoxybenzamine and 3.345 ng/mL phenoxybenzamine-d5 and was equivalent to extracted QC HIGH assuming 100% recovery. The QC Medium Neat Solution contained 0.801 ng/mL phenoxybenzamine and 3.345 ng/mL phenoxybenzamine-d5 and was equivalent to extracted QC MED assuming 100% recovery. The QC Low Neat Solution contained 0.100 ng/mL phenoxybenzamine and 3.345 ng/mL phenoxybenzamine-d5 and was equivalent to extracted QC LOW assuming 100% recovery. The “Recovery QCs” and “Reference QCs” samples were analyzed. Recovery percentages for phenoxybenzamine and the internal standard were calculated by comparing the absolute peak response of an extracted sample to the mean peak response of non-extracted (neat) reference solution prepared at three QC levels (QC LOW, QC MED, and QC HIGH) assuming 100% recovery. The % coefficient of variation of recovery was calculated for each analyte and internal standard. The results are summarized in Table III.

### Determination of Assay Accuracy and Precision

The accuracy and precision of the assay was determined by measuring concentrations of phenoxybenzamine at different concentration levels in replicates of six, and calculating standard deviation, coefficient of variance, and relative error.

TABLE IV
Determination of assay accuracy and precision.

| | LLOQ | QC LOW | QC MED | QC HIGH |
|---|---|---|---|---|
| | (0.200 ng/mL) | (0.601 ng/mL) | (4.808 ng/mL) | (19.232 ng/mL) |
| | 0.199 | 0.631 | 4.678 | 18.635 |
| | 0.218 | 0.607 | 4.540 | 18.645 |
| | 0.206 | 0.588 | 4.629 | 18.787 |
| | 0.207 | 0.618 | 4.732 | 18.712 |
| | 0.210 | 0.591 | 4.888 | 18.775 |
| | 0.201 | 0.616 | 4.844 | 18.562 |
| Mean | 0.207 | 0.609 | 4.719 | 18.686 |
| SD | 0.007 | 0.017 | 0.131 | 0.088 |
| % CV | 3.3 | 2.7 | 2.8 | 0.5 |
| % RE | 3.5 | 1.2 | −1.9 | −2.8 |
| n | 6 | 6 | 6 | 6 |

### Stability of Phenoxybenzamine in Whole Blood

Stability of phenoxybenzamine in human whole blood was determined by spiking phenoxybenzamine into fresh human whole blood. This was followed by the preparation of the whole blood samples into two sets: one set was immediately processed into plasma and the other set was stored at 4° C.±4° C. for up to two hours and then processed into plasma. Samples were extracted in replicates of six with control blank and analyzed. The results are summarized in Table V. The results show that phenoxybenzamine is stable in whole blood for up to 2 hours when stored at 4° C.±4° C.

TABLE V
Stability of phenoxybenzamine in whole blood (“WB”).

| | WB Sample |
|---|---|
| Time (hours) | Amount Found (ng/mL) |
| 0.0 | 3.381 |
| | 3.387 |
| | 3.305 |
| | 3.365 |
| | 3.379 |
| | 3.325 |
| Mean | 3.357 |
| ~2 | 3.315 |
| | 3.330 |
| | 3.365 |
| | 3.328 |
| | 3.283 |
| | 3.160 |
| Mean | 3.297 |
| % Difference | −1.8 |

### Autosampler Re-Injection Reproducibility

Autosampler re-injection reproducibility for phenoxybenzamine was demonstrated by having extracted QC samples (QC LOW and QC HIGH) analyzed. The mean concentration of the aged re-injected samples was compared with the mean concentration of the initial zero hour samples prepared at the same concentration as the QC samples. Table VI summarizes the results. The results show that phenoxybenzamine, in extracted samples, can be re-injected for up to 110 hours when stored in autosampler at 7° C.±4° C.

TABLE VI
Autosampler re-injection reproducibility of
phenoxybenzamine at 7° C. ± 4° C. for 110 hours.

| | Amount Added (ng/mL) | |
|---|---|---|
| | QC LOW | QC HIGH |
| | 0.601 | 19.232 |
| Time (hours) | Amount Found (ng/mL) | |
| 0.0 | 0.610 | 18.161 |
| | 0.590 | 18.305 |
| | 0.603 | 18.069 |
| Mean | 0.601 | 18.178 |
| ~75 | 0.610 | 18.066 |
| | 0.646 | 18.686 |
| | 0.590 | 18.482 |
| Mean | 0.615 | 18.411 |
| % Difference | 2.4 | 1.3 |
| ~110 | 0.602 | 18.179 |
| | 0.612 | 18.435 |
| | 0.595 | 18.656 |
| Mean | 0.603 | 18.423 |
| % Difference | 0.3 | 1.3 |

### Processed Sample Integrity

Processed sample integrity for phenoxybenzamine was demonstrated by having processed QC samples (QC LOW and QC HIGH) analyzed after a duration of time equivalent to the time samples were expected to sit in storage prior to autosampler injection without affecting the integrity of the samples. The mean concentration at each time-point was compared with the mean concentration of the zero hour samples. The results are summarized in Table VII. It shows that the integrity of the samples for phenoxybenzamine, at LOW and HIGH concentrations, is not affected for up to 112 hours at 4° C.±4° C.

TABLE VII
Processed sample integrity of phenoxybenzamine
at 4° C. ± 4° C. for 112 Hours.

| | Amount Added (ng/mL) | |
|---|---|---|
| | QC LOW | QC HIGH |
| | 0.601 | 19.232 |
| Time (hours) | Amount Found (ng/mL) | |
| 0.0 | 0.610 | 18.161 |
| | 0.590 | 18.305 |
| | 0.603 | 18.069 |
| Mean | 0.601 | 18.178 |
| ~112 | 0.574 | 18.209 |
| | 0.590 | 18.214 |
| | 0.586 | 18.220 |
| Mean | 0.583 | 18.214 |
| % Difference | −2.9 | 0.2 |

### Example 2

### Evaluating Stability of Phenoxybenzamine

### Stability of Phenoxybenzamine in Neat Solution

The stability of phenoxybenzamine in neat solutions was examined. The measured amount of phenoxybenzamine in neat solution dropped ˜90% in 24 hours at ambient temperature but ˜20% at 4° C.

### Stability of Phenoxybenzamine in Various Solvents

Various solvents were examined for their abilities to stabilize phenoxybenzamine. The solvents examined were alcohol, acetonitrile (ACN), methanol (MeOH), acidified solvents (e.g., acidified MeOH and acidified ACN). Phenoxybenzamine in these solvents was not stable for 24 hr at 4° C. The stability of phenoxybenzamine was examined in acidified ACN with 0.5%, 1% and 2% formic acid (“FA”). Phenoxybenzamine was stable for ˜3 hr at RT and 4° C. The measured amount of phenoxybenzamine for neat standard in 0.5% FA in ACN was higher than those using 1% FA in ACN, and 2% FA in ACN. The autosamper stability at ˜24 hr for phenoxybenzamine in 0.5% FA in ACN was found to drop by ˜70%.

The stability of phenoxybenzamine was examined in acidified MeOH (80% MeOH containing 0.1% TFA). Phenoxybenzamine was stable for at least 48 hours at 4° C., −25° C. and −70° C.

### Stability of Phenoxybenzamine in Plasma

The stability of phenoxybenzamine in acidified plasma samples containing 1% FA, 1% H₃PO4, 2% H₃PO4 or 0.1N HCl was tested. All seemed to be stable except for 1% FA. Autosampler re-injection stability of phenoxybenzamine in 80% MeOH containing 0.1% TFA was found to be stable for at least 24 hours.

Therefore, acidified solvent containing 80% MeOH and 0.1% TFA was able to stabilize phenoxybenzamine. Lower temperature (e.g., 4° C. or lower) also helped stabilize phenoxybenzamine. Acidified plasma was able to stabilize phenoxybenzamine, and the stronger acid, H₃PO₄, was the most optimal for stabilizing phenoxybenzamine in plasma. Samples kept at 4° C. during extraction also helped to stabilize phenoxybenzamine.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

The disclosures of all publications, patents, patent applications and published patent applications referred to herein by an identifying citation are hereby incorporated herein by reference in their entirety.

What is claimed is:

1. A method for preparing an extract from a biological sample suitable for determining concentration of a basic or neutral compound, comprising the step of extracting the compound from the biological sample using an acidic alcohol-water mixture.

2. A method for determining the concentration of a basic or neutral compound in a biological sample, comprising the steps of:
(a) extracting the compound from the biological sample using an acidic alcohol-water mixture, whereby an extract comprising the compound is obtained, and
(b) analyzing the extract to determine the concentration of the compound.

3. A method for determining the concentration of a basic or neutral compound in a biological sample, comprising the steps of:
(a) extracting the compound from the biological sample, whereby an extract comprising the compound is obtained, wherein the extract comprises at least about 50% of the compound from the biological sample, and
(b) analyzing the extract to determine the concentration of the compound.

4. The method of claim 3, wherein the extract comprises at least about 70% of the compound from the biological sample.

5. The method of claim 4, wherein the extract comprises at least about 90% of the compound from the biological sample.

6. A method for determining the concentration of a basic or neutral compound in a biological sample comprising less than about 150 ng of the compound in 1 mL of the biological sample, comprising the steps of:
(a) extracting the compound from the biological sample, whereby an extract comprising the compound is obtained, and
(b) analyzing the extract to determine the concentration of the compound.

7. The method of claim 6, wherein the concentration of the basic or neutral compound in the biological sample comprises less than about 50 ng of the compound in 1 mL of the biological sample.

8. The method of claim 6, wherein the concentration of the basic or neutral compound in the biological sample comprises less than about 20 ng of the compound in 1 mL of the biological sample.

9. The method of claim 6, wherein the concentration of the basic or neutral compound in the biological sample comprises less than about 10 ng of the compound in 1 mL of the biological sample.

10. The method of claim 6, wherein the concentration of the basic or neutral compound in the biological sample comprises less than about 1 ng of the compound in 1 mL of the biological sample.

11. The method of any one of claims 1-10, wherein the compound is phenoxybenzamine.

12. The method of any one of claims 1-10, wherein the compound is a modified phenoxybenzamine, a derivative of phenoxybenzamine, a degradation product of phenoxybenzamine, or a metabolite of phenoxybenzamine.

13. The method of any one of claims 1-12, wherein the biological sample is a biological liquid sample.

14. The method of any one of claims 1-12, wherein the biological sample is plasma.

15. The method of any one of claims 1-12, wherein the biological sample is urine.

16. The method of any one of claims 1-12, wherein the biological sample is an extract from biological liquid sample or biological tissue sample.

17. A method for measuring the concentration of a neutral or basic compound in a blood plasma sample, comprising the steps of:
(a) extracting the compound from the blood plasma sample using an acidic alcohol-water mixture, whereby an alcohol extract comprising the compound is obtained, and
(b) analyzing the extract to determine the concentration of the compound.

18. A method for stabilizing a basic or neutral compound comprising combining a composition comprising the basic or neutral compound with an acidic alcohol-water mixture, wherein stability of the basic or neutral compound is enhanced as compared to a composition without the acidic alcohol-water mixture.

19. The method of claim 17 or 18, wherein the compound is phenoxybenzamine.

20. The method of claim 17 or 18, wherein the compound is a modified phenoxybenzamine, a derivative of phenoxybenzamine, a degradation product of phenoxybenzamine, or a metabolite of phenoxybenzamine.

21. The method of any one of claims 18-20, wherein the composition comprises the compound at a concentration of about 1 ng/mL to about 1 g/mL.

22. The method of claim 21, wherein the composition comprises the compound at a concentration of about 20 ng/mL to about 100 mg/mL.

23. The method of claim 22, wherein the composition comprises the compound at a concentration of 20 ng/mL to 1 mg/mL.

24. The method of any one of claims 3-16, wherein the step of extracting the compound is performed using an acidic alcohol-water mixture.

25. The method of any one of claims 1-24, wherein the acidic alcohol-water mixture comprises about 0.1% trifluoroacetic acid.

26. The method of any one of claims 1-25, wherein the alcohol is methanol or ethanol.

27. The method of any one of claims 1-26, wherein the acidic alcohol-water mixture comprises at least about 20% alcohol.

28. The method of claim 27, wherein the acidic alcohol-water mixture comprises at least about 50% alcohol.

29. The method of claim 28, wherein the acidic alcohol-water mixture comprises at least about 75% alcohol.

30. The method of any one of claims 1-17, 19, 20, and 24-29, wherein the extracting step is performed by solid phase extraction.

31. The method of any one of claims 2-17, 19, 20, and 24-29, wherein the step of analyzing the extract is performed using liquid chromatography.

32. The method of claim 31, wherein the step of analyzing the extract is performed using high performance liquid chromatography.

33. The method claim 31, wherein the step of analyzing the extract is performed using LC-MS/MS.

34. The method of any one of claims 1-17, 19, 20, and 24-33, wherein the biological sample is obtained from a patient treated with phenoxybenzamine.

35. The method of claim 11, wherein the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol does not significantly affect the accuracy of the measurement of the concentration of phenoxybenzamine.

36. The method of claim 11, wherein the presence in the biological sample of one or more compounds selected from caffeine, ibuprofen, salicylic acid, acetylsalicylic acid, acetaminophen, theophylline, dextromethorphan, loratadine, dimenhydrinate, nicotine, desogestrel, cyproterone acetate, levonorgestrel, norgestimate, and ethinyl estradiol does not significantly affect the selectivity of the method used in the measurement of phenoxybenzamine in human plasma.

37. A method of treating a patient comprising the steps of
(a) administering phenoxybenzamine to the patient,
(b) measuring the concentration of phenoxybenzamine in a biological sample obtained from the patient, and
(c) based on the concentration of phenoxybenzamine in the biological sample, administering appropriate amount of another drug to achieve a therapeutic benefit in the patient.

38. The method of claim 38, wherein the biological sample is blood plasma.
